(19) United States
(12) Patent Application Publication
(10) Pub. No.: US 2015/0252370 A1
(43) Pub. Date: Sep. 10, 2015

(54) USE OF ATX INHIBITORS FOR TREATMENT OR PREVENTION OF INFLUENZA VIRUS A INFECTIONS

(71) Applicant: Academia Sinica, Taipei (TW)

(72) Inventors: Michael M. C. LAI, New Taipei City (TW); Hsu-Ling CHIEN, New Taipei City (TW); King-Song JENG, Taipei (TW); Wen-Chi SU, Taichung City (TW)

(73) Assignee: Academia Sinica, Taipei (TW)

(21) Appl. No.: 14/196,365

(22) Filed: Mar. 4, 2014

(51) Int. Cl. C12N 15/113; A61K 31/502; C07K 16/40

(52) U.S. Cl. CPC C12N 15/1138; C07K 16/40; A61K 31/502; C12N 2320/30

(57) ABSTRACT

The present invention relates to a method for treating or preventing an influenza virus type A infection, particularly by using an ATX inhibitor.

### TECHNOLOGY FIELD

The present invention relates to a method for treating or preventing an influenza virus type A infection, particularly by using an ATX inhibitor.

### BACKGROUND OF THE INVENTION

The Influenza A virus (IAV), a member of the family Orthomyxoviridae, is an enveloped, negative-sense RNA virus containing an eight-segmented genome. The IAV is an obligate intracellular pathogen, relying on host cell proteins and hijacking host machinery to complete viral life cycle and for propagation. The consequence of the IAV entry into the host cell is to release the eight viral genome segments into the nucleus to initiate virus transcription and replication. These eight genome segments encode ten viral proteins, including two envelope glycoproteins, hemagglutinin (HA) and neuraminidase (NA), three polymerase subunits of viral-specific RNA polymerase, polymerase acidic protein (PA), polymerase basic protein 1 and 2 (PB1 and PB2), the nucleoprotein (NP), and the matrix protein 1 (M1), ion channel protein M2, and two nonstructural proteins 1 and 2 (NS1 and NS2), which are the products by using alternative reading frames of the same RNA segment. IAV infection causes severe respiratory and/or intestinal illnesses in a variety of animal hosts, such as birds and mammals. The global burden of IAV infections poses major public health problems worldwide. Current influenza vaccines require annual updating and provide only partial protection. In addition, there are only two categories of drugs available, the M2 proton channel inhibitors (amantadine) and the neuraminidase inhibitors (oseltamivir). However, the drug resistance against these drugs has increased worldwide. Thus, there is an urgent need to search for a new antiviral agent to combat the IAV infection.

Sialic acid has long been considered to be the receptor for influenza virus. The first stage of influenza virus entry is recognition of a sialic acid-containing receptor molecule by viral HA. The HA of human influenza A viruses prefers −2,6-linked sialic acid-containing glycoproteins, but HA of avian influenza A viruses prefers −2,3-linked sialic acid glycoproteins, thus accounting for the difference in the host specificity of theses virus strains (Kogure et al., 2006; Suzuki et al., 2001). However, it is not clear whether influenza virus preferentially utilize certain protein receptors or not. Such a preference will restrict or expand host range or species specificity of the virus. An entire virus entry process can be divided into several steps. At first step, the glycoprotein HA of influenza virus particle recognizes a specific receptor molecule on the cell surface. The receptor molecule of influenza virus is a terminal sialic acid residue that is linked to saccharides anchored on the cell surface membrane. In the second step, the cell endocytosis is induced to generate an endosome to encapsulate the influenza virus particle. In the third step, the endosome is translocated to a site near the cell nucleus. At fourth step, the viral membrane fuses with the host membrane; the fusion step is mediated by the HA glycoprotein embedded in the virus envelope. After the fusion step, the eight segments of RNA genome are released to the nucleus, and the viral transcription and replication are initiated (Lakadamyali et al., 2003; Lakadamyali et al., 2004; Luo 2012; Rust et al., 2004). However, the cell biological aspects of influenza virus entry process and the additional host factors involved remain unclear.

Anexelekto (AXL) is a member of the TAM (Tyro 3, AXL, Mer) protein family, and is a receptor tyrosine kinase (RTK), which is a single-pass type 1 transmembrane protein. AXL is expressed ubiquitously in many human tissues and cancer cell lines, such as normal human bronchial epithelia cells (NHBE) and human lung cancer cell lines (Brindley et al., 2011; Wimmel et al., 2001), lung, platelets, monocytes/macrophages, and heart (Angelillo-Scherrer et al., 2001; Neubauer et al., 1994; O'Bryan et al., 1991). The N-terminal ectodomain of AXL consists of two immunoglobulin-like (Ig) domains and two fibronectin-type III (FNIII) domains. The C-terminal cytoplasmic domain of AXL contains tyrosine kinase domains (Linger et al., 2008). The two Ig-like domains are responsible for TAM interactions with its ligands, growth-arrest-specific gene 6 (Gas6) and Protein S (Stitt et al., 1995; Varnum et al., 1995).

According to typical activation of RTKs, the ligand binding to the extracellular domain of AXL may lead to homo- or heterodimerization of AXL and subsequently induce trans-autophosphorylation of tyrosine residues within the cytoplasmic domain. The tyrosine-phosphorylated AXL recruits signaling molecules and further activates downstream signaling pathways. The tyrosine-phosphorylated 779 and 821 of AXL can recruit and activate growth factor receptor-bound protein 2 (GRB2) and the p85 subunit of phosphatidylinositol-3 kinase (PI3 kinase), and lead to downstream phosphorylation of Akt at threonine 308 (Braunger et al., 1997; Weigner et al., 2008). The AXL signaling pathways result in a variety of cell type-dependent effects, including platelet aggregation (Wang et al., 2007), cell survival (Zheng et al., 2009), proliferation (Fridell et al., 1996), regulation of proinflammatory cytokine production (Lu et al., 2001), and regulation of actin cytoskeleton (Nielsen-Preiss et al., 2007).

Recently, the AXL was found to facilitate the virus entry of Zaire ebolavirus (ZEBOV), Lake Victoria marburgvirus (MARV), lentivirus vectors, and vaccinia virus (Mercer et al., 2011; Morizono et al., 2011; Shimojima et al., 2006). However, the role of AXL in IAV infection has not been reported.

### BRIEF SUMMARY OF THE INVENTION

In this study, we found that AXL is a novel receptor in IAV replication. IAV replication was decreased in cells deprived of AXL and enhanced in cells over-expressing AXL. The IAV attachment was decreased by treatment with a polyclonal antibody against cell surface AXL during virus entry stage. The IAV binding to the cell surface was also decreased by knockdown of AXL. The AXL phosphorylation was induced upon IAV infection. Furthermore, an AXL kinase inhibitor, R428, blocked virus infection at the virus entry step. Our findings indicated that AXL contributes to an entry stage of IAV infection in a sialic acid-dependent manner. We also examined the interactions between AXL and envelope proteins by co-immunoprecipitation assay. We found that AXL binds with the neuraminidase (NA), M2 and hemagglutinin (HA). This property of AXL is reminiscent of the sialic acid receptor. These results suggest that blockage of AXL is a potential new avenue for developing an antiviral therapy against IVA infection.

Accordingly, the present invention provides a method for the treatment or prevention of an influenza virus type A infection which comprises administering to a subject a therapeutically effective amount of an AXL inhibitor to a subject.

The present invention also provides use of an AXL inhibitor in the manufacture of a medicament for the treatment or prevention of an influenza virus type A infection in a subject.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows that the human AXL was required for efficient infection of IAV. A549 cells were transduced with lentivirus carrying AXL shRNA clone #1, #2, or control shLacZ. (A) The knockdown effect of AXL was examined by immunoblotting with AXL-specific antibody. A549-shAXL clone #1, #2, or shLacZ were infected with IAV (MOI of 0.5) for 8 h. The IAV replication levels were analyzed by (A) immunoblotting with NP antibody and (B) real-time RT-PCR by using the primer specific to IAV-NP. Results are means and standard deviations of two independent experiments. The ratio of the indicated groups were compared by two-tailed Student's t test (n=2), and the results are shown (*, P<0.05). (C) The human T-RE-x-293 with inducible expression of human AXL clone #1, #2, or vector were cultured in medium without (−) or with (+) Dox (1 μg/ml) for 12 h, then were infected with IAV (MOI of 0.5) for 8 h. Extracts were subjected to immunoblotting using antibodies against AXL, IAV-NP, and actin as indicated.

FIG. 2 shows that the cell surface of AXL was required for IAV infection. (A) A549 cells were treated with medium (mock), anti-AXL (25 μg/ml), preimmune control antibody (25 μg/ml, control Ab), or sialidase (0.01 U/ml) at the indicated times before, during, or post-infection with IAV (MOI of 0.2) for 17 h. (B) The cell lysates were harvested for immunoblotting with antibodies against IAV-NP, -NS1, or actin, as indicated. (C) A549 cells were pretreated with medium (mock), anti-AXL (25 μg/ml), or preimmune control antibody (25 μg/ml, control Ab) at 4° C. for 2 h. The IAV (MOI of 0.2) were adsorbed at 4° C. for 1 h in the presence of antibodies. After adsorption, the cells were washed and further incubated at 37° C. for 17 h. The cell lysates were collected for immunoblotting with antibodies against IAV-NP, -NS1, or actin, as indicated. (D) The A549-shAXL clone #1, #2, and control shLacZ cells were adsorbed with IAV (MOI of 5) at 4° C. for 1 h, and PBS wash (pH 7, 4° C.) was performed to remove unattached virus before cell lysis. The attached virus particles were visualized with a M1 monoclonal antibody by immunoblotting analysis.

FIG. 3 shows that AXL kinase activity was induced upon IAV infection and required for efficient uptake of IAV particles. (A) and (B) The T-RE-x-293/human AXL cells were cultured in medium with (+) Dox (1 μg/ml) for 12 h, and mock-infected, or infected with IAV (MOI of 5) for 15, 30, 45, or 60 min. For positive control, cells were treated with Gas6 (100 ng/ml) for 30 min. For negative control, cells were pretreated with AXL inhibitor (8 μM of R428) or solvent control (DMSO) for 1 h at 37° C. and then stimulated by Gas6 (100 ng/ml) for 30 min. The cell lysates were examined by immunoblotting analysis with the antibodies against AXL (Tyr702) tyrosine phosphorylation, AXL, IAV-M1, and actin as indicated. (C) A549 cells were treated with medium (mock), solvent control (DMSO), or AXL kinase inhibitor (8 μM of R428) at the indicated times before, during, or post-infection with IAV (MOI of 0.5) for 8 h. The cell lysates were harvested and analyzed by immunoblotting with antibodies against IAV-NP, -NS1, and actin.

FIG. 4 shows that IAV attachment promoted by AXL is a sialic acid dependent manner. The T-RE-x-293/human AXL were cultured with Dox (1 μg/ml) for induction of AXL expression. (A) Cells were pretreated without (−) or with (+) sialidase (0.01 U/ml) at 37° C. for 1 h and adsorbed with IAV (MOI of 5) at 4° C. for 1 h. The unattached virus was removed by PBS wash (pH 7, 4° C.). Western blotting was performed by antibodies against IAV-M1, AXL, and actin. (B) Cells without (−) or with (+) sialidase treatment were mock-infected, infected with IAV (MOI of 5) for 15 and 30 min, or Gas6 (100 ng/ml) treatment for 30 minutes. The cell lysates were analyzed by immunoblotting with antibodies against AXL (Tyr702) tyrosine phosphorylation, AXL, IAV-M1, or actin. (C) Cells treated without (−) or with (+) sialidase were infected with IAV (MOI of 0.5) for 8 h. The cell lysate were collected and analyzed by immunoblotting using antibodies against IAV-NP, -NS1, and actin.

FIG. 5 shows that the interactions between IAV and envelope proteins of IAV. The T-RE-x-293/human AXL cells were transfected with individual plasmids encoding HA-tagged NP, HA, NA, M2, or mCherry and also treated with Dox (1 μg/ml) for induction of AXL expression. (A) The whole-cell lysates were immunoprecipitated with anti-HA-agarose overnight at 4° C. (B) For immunoprecipitation by anti-AXL, the cell lysates were immunoprecipitated by anti-AXL overnight at 4° C. and the immunocomplex were further captured by Protein A/G at 4° C. for 3 h. The precipitates were washed by wash buffer (1×TBS with 0.1% Tween-20) and proteins were analyzed by immunoblotting with antibodies against HA-tagged viral proteins and AXL.

### DETAILED DESCRIPTION OF THE DRAWING

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

In the present invention, we investigated the role of AXL in IAV infection and found that AXL is a cellular factor required for IAV infection. We showed that treatment with an antagonist polyclonal antibody against AXL completely blocked virus infection, and R428, a selective small-molecule inhibitor of AXL kinase, which has been reported to block tumor spread and prolong survival in mouse models of metastatic breast cancer, also significantly inhibited influenza A virus infection at the entry stage of infection. Thus, blockage of AXL is a potential new target for developing antiviral therapy against IAV infection.

Therefore, the present invention provides a method for treatment or prevention of an influenza virus type A infection by administering an AXL inhibitor to a subject in need thereof.

As used herein, the term “influenza virus type A infection” refers to any infection caused by an influenza virus type A. The subtypes of influenza virus type A are determined based on the combination of the virus envelope glycoproteins hemagglutinin (HA) and neuraminidase (NA) subtypes. There are 16 different HA antigens (H1-H16) and nine different NA antigens (N1-N9) for influenza virus type A. Exemplary influenza virus type A include but are not limited to H1N1, H2N2, H3N2, H5N1, H7N7, H1N2, H9N2, H7N2, H7N3, and H10N7. In one certain embodiment, the subtype of influenza virus type A according to the present invention is H1N1.

As used herein, the term “treating” or “treatment” at least includes curing, healing, alleviating, relieving, remedying, ameliorating, improving or affecting a disease or condition, the symptoms of the disease or condition, or the complications of the disease or condition in a subject. As used herein, the term “preventing” “prevention” or “prophylactic treatment” at least includes reduction of likelihood or susceptibility to acquiring a disease or disorder or a predisposition to develop the disease or disorder in a subject. Therefore, a subject in need of the treatment or prevention of influenza virus type A infection according to the present invention includes those already diagnosed or determined as having the influenza virus type A infection as well as those susceptible or predisposed to such infection. Influenza virus infection can be diagnosed or determined by any standard approach or method as known in the art, including, but not limited to, detection of flu symptoms and measurement of viral titers or specific viral nucleic acids or antigens. Particularly, “treating” or “treatment” or “preventing” “prevention” or “prophylactic treatment” can be conducted by the application or administration of a composition including one or more active agents to a subject in need with the purpose as above. In some embodiments of the invention, an antiviral agent can be administered to a subject after the virus infection for the treatment purpose or prior to the virus infection for the prophylaxis purpose.

As used herein, the term “Anexelekto receptor” or “AXL receptor” or “AXL” interchangeably refer to a member of the receptor tyrosine kinase subfamily. The AXL receptor has a unique structure which comprises an N-terminal ectodomain (extracellular region) containing two immunoglobulin-like (Ig) domains and two fibronectin-type III (FNIII) domains, and a C-terminal cytoplasmic domain containing tyrosine kinase domains. The two Ig-like domains are responsible for the interaction with its ligands, such as growth-arrest-specific gene 6 (Gas6) and protein S, which induces downstream signaling pathways resulting in a variety of cell type-dependent effects, such as platelet aggregation (Wang et al., 2007), cell survival (Zheng et al., 2009), proliferation (Fridell et al., 1996), regulation of proinflammatory cytokine production (Lu et al., 2001) and regulation of actin cytoskeleton (Nielsen-Preiss et al., 2007). The AXL gene is evolutionarily conserved between mammal species. For example, the AXL gene among human and mouse have at least 80% sequence identity. Typically, the AXL receptor as described herein can be from mammals e.g. humans or non-human mammals. The nucleotide and amino acid sequences of the AXL receptor are known in the art, for example, the human AXL receptor, as described in O'Bryan et al. 1991, or as published as GenBank accession number NM_{—}021913.4 (SEQ ID NO: 1) and NP_{—}068713.2 (SEQ ID NO: 2). In some certain embodiments, the AXL receptor has an amino acid sequence having at least 80%, 85%, 90%, or 95% identity to SEQ ID NO: 2.

As used herein, the term “AXL inhibitor” refers to an agent, compound or substance which is capable of inhibiting the function or activity of the AXL receptor, for example, by binding or not to the AXL receptor. Particularly, the AXL inhibitor as used herein can be a small molecule organic compound, an antibody and a polynucleotide. A suitable AXL inhibitor according to the present invention can be identified by persons skilled in the art using various known methods, for example, by its ability to bind to the AXL receptor and inhibit the kinase activity or by its ability to block or reduce the gene expression of the AXL receptor. Specifically, the AXL inhibitor of the invention blocks the phosphorylation of AXL at amino acid T702 and/or Y779. In some embodiments, the AXL kinase activity or expression level is reduced, by about 10% less, about 20% less, about 30% less, about 40% less, about 50% less, about 60% less, about 70% less, about 80% less, about 90% less, or completely blocked by an AXL inhibitor of the present invention, as compared with a control AXL not exposed to the AXL inhibitor.

In some embodiments, the AXL inhibitor of the invention is an AXL antagonist which is capable of specifically binding to the AXL receptor and inactivating, fully or partially, the AXL activity. In certain embodiments, an AXL antagonist of the invention is a small organic molecule. As used herein, the term “small organic molecule” is recognized in the art and refers to a molecule of a size comparable to those organic molecules generally used in pharmaceuticals, which does not include biological macromolecules such as proteins or nucleic acids. Preferred small organic molecules are characterized as having a size less than 10,000 Da, more preferably less than 5,000 Da, even more preferably less than 2,000 Da, and most preferably less than 1,000 Da. In one certain embodiment, an AXL antagonist of the invention is 1-(6,7-dihydro-5H-benzo[6,7]cyclohepta[1,2-c]pyridazin-3-yl)-N3-(7-(pyrrolidin-1-yl)-6,7,8,9-tetrahydro-5H-benzo[7]annulen-2-yl)-1H-1,2,4-triazole-3,5-diamine, namely R428.

In other embodiments, the AXL inhibitor of the invention is an antagonist antibody that specifically binds to AXL and inactivating, fully or partially, the AXL activity.

As used herein, the term “antibody” refers to an intact immunoglobulin or fragment thereof, and includes any polypeptide comprising an antigen-binding domain or an antigen-binding fragment that specifically binds to a particular antigen. The term includes but is not limited to monoclonal, polyclonal, humanized, human, single-chain, chimeric, synthetic, recombinant and hybrid antibodies.

As used herein, the term “specific binds” or “specifically binding” refers to a non-random binding reaction between two molecules, such as the binding of the antibody to an epitope of the antigen. The affinity of the binding is defined by the terms ka (associate rate constant), KD (equilibrium dissociation) or kd (dissociation rate constant). Typically, binding or specifically binding when used with respect to an antibody refers to an antibody which specifically binds to (or “recognizes”) its target(s) with an affinity (KD) value less than less than 10⁻⁸ M, particularly less than 10⁻⁹ M. A lower KD value represents a higher binding affinity (i.e. stronger binding) so that a KD value of 10⁻⁹ represents a higher binding affinity than a KD value of 10⁻⁸.

The term “antigen-binding domain” or “antigen-binding fragment” refers to a portion or region of an entire antibody molecule that is responsible for antigen binding. The portion of the antigen that is specifically bound or recognized by the antibody is called the “epitope.” An antigen-binding domain may comprise the heavy chain variable region (V_{H}) and the light chain variable region (V_{L}); however, it does not have to comprise both. The variable region in both chains typically contains three hypervariable regions called the complementarity determining regions (CDRs). The three CDRs are interrupted by framework regions (FRs), which are more highly conserved than the CDRs. The constant regions of the heavy and light chains are not responsible for antigen binding, but exhibit various effector functions. Antibodies are classified based on the amino acid sequence of the constant region of their heavy chain. The five major classes or isotypes of antibodies are IgG, IgM, IgA, IgD and IgE, which are characterized by the presence of the constant regions of the heavy chains, gamma, mu, alpha, delta and epison, respectively. Examples of antigen-binding fragments of an antibody include: (1) a Fab fragment, a monovalent fragment having the V_{L}, V_{H}, C_{L} and C_{H}1 domains; (2) a F(ab′)₂ fragment, a bivalent fragment having two Fab fragments linked by a disulfide bridge at the hinge region, i.e. a dimer of Fab; (3) a Fv fragment having the V_{L} and V_{H} domains of a single arm of an antibody; (4) an isolated complementarity determining region (CDR); (5) a single chain Fv (scFv), a single polypeptide chain composed of a V_{H} domain and a V_{L} domain through a peptide linker; and (6) a (scFv)₂, comprising three peptide chains: two V_{H} domains linked by a peptide linker and bound by disulfide bridges to two V_{L} domains.

Numerous methods known to those skilled in the art are available for obtaining desired antibodies. For example, antibodies can be produced using recombinant DNA methods. Monoclonal antibodies may also be produced by generation of hybridomas. Hybridomas formed in this manner are then screened using standard methods, such as enzyme-linked immunosorbent assay (ELISA) to identify one or more hybridomas that produce an antibody that specifically binds with a specified antigen. In addition, phage display systems can be used to screen for single chain antibodies.

In some embodiments, the AXL antagonist antibody of the invention specifically recognizes the extracellular domain of AXL, such as the amino acid residues 33-440 of human AXL (SEQ ID NO: 2).

In another embodiment of the invention, the AXL inhibitor of the invention is an inhibitory polynucleotide which is capable of suppressing AXL expression. Such AXL inhibitory polynucleotide of the invention include a short interfering RNA (siRNA), synthetic hairpin RNA (shRNA) or anti-sense nucleic acids.

The term “polynucleotide” or “nucleic acid” refers to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated synthesizer. It will be understood that when a nucleotide sequence is represented by a DNA sequence (i.e., A, T, G, C), this also includes an RNA sequence (i.e., A, U, G, C) in which “U” replaces “T.”

The term “complementary” refers to the topological compatibility or matching together of interacting surfaces of two polynucleotides. Thus, the two molecules can be described as complementary, and furthermore the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. For example, the polynucleotide whose sequence 5′-TATAC-3′ is complementary to a polynucleotide whose sequence is 5′-GTATA-3′.”

In some embodiments, suppression of AXL expression by an inhibitory polynucleotide according to the invention is mediated by RNA interference (RNAi). RNA interference (RNAi) has been shown effective in silencing or knocking down expression of a target gene in various organisms. Typically, it is accomplished by selective inactivation of the corresponding mRNA of a target gene by using double-stranded RNAs (siRNA or shRNA). The term “small interfering RNA (siRNA)” refers to an RNA or RNA analog comprising typically less than 100 base pairs and can be about 30 base pairs or shorter such as 29 bps, 25 bps, 23 bps, 21 bps, 20 bps, 15 bps, 10 bps or any integer thereabout or therebetween. Such siRNA in cells is unwounded into two single stranded (ss) RNAs, i.e. the passenger strand and the guide strand, wherein the passenger strand is then degraded and the guide strand is incorporated into the RNA-induced silencing complex (RISC) where the gene silencing occurs when the guide strand pairs with a complementary sequence in a mRNA molecule of a target gene and induces cleavage. siRNA can be either chemically synthesized and then transfected into cells or can be produced inside the cells by introducing vectors (such as a lentiviral vector) that express short-hairpin RNA (shRNA) as a precursor of siRNAs. In addition to RNA interference by double stranded RNAs, other polynucleotides targeting AXL can be used in the practice of the present invention, such as antisense RNA or DNA molecules. Antisense RNA or DNA molecules are single stranded which are complementary to a portion of a specific target mRNA molecule such that a double stranded molecule is formed and the translation process is inhibited. Methods and tools for designing inhibitory polynucleotide targeting a gene are known and available in the art.

The present invention provides a method for the present invention provides a method for treatment or prevention of an influenza virus type A infection by administering an AXL inhibitor to a subject in need thereof.

As used herein, the term “subject” or “patient” refers to humans or non-human mammals such as companion animals (e.g., dogs, cats, and the like), farm animals (e.g., cows, sheep, pigs, horses, and the like) or laboratory animals (e.g., rats, mice, guinea pigs, and the like).

In particular, an AXL inhibitor of the present invention is administered in a therapeutically effective amount. As used herein, the term “therapeutically effective amount” refers to an amount of a drug or pharmaceutical agent which, as compared to a corresponding subject who has not received such amount, results in an intended pharmacological result, or an effect in treatment, healing, prevention, or amelioration of a disease or disorder (e.g. influenza infection), or a decrease in the rate of advancement of a disease or disorder. The effective amount or dose of a pharmacological agent may vary depending on particular active ingredient employed, the mode of administration, and the age, size, and condition of the subject to be treated. Precise amounts of a pharmacological agent are required to be administered depend on the judgment of the practitioner and are peculiar to each individual.

According to the invention, an AXL inhibitor can be administered to a subject already suffering from an influenza virus type A infection for the treatment purpose, or a subject not already suffering from an influenza virus type A infection but at the risk of, or having a predisposition, to develop such an infection for the preventive or prophylaxis purpose. In certain embodiments, the AXL inhibitor is administered prior to infection or at a time after infection, preferably immediately, or immediately after appearance of symptoms of infection. For example, in some embodiments, the AXL inhibitor may be administered about 6 hours, 12 hours, one day, two days, three days, four days or five days before infection. In some embodiments, the AXL inhibitor may be administered within 1-12 hours, within one days, within two days, within three days, within four days or within five days after likelihood of an infection or emergence of symptoms of the viral infection.

Preferably, for delivery purpose, an AXL inhibitor according to the present invention is formulated with a pharmaceutically acceptable carrier to form a pharmaceutical composition. As used herein, the term “pharmaceutically acceptable” means that the carrier is compatible with the active ingredient contained in the composition, preferably capable of stabilizing the active ingredient, and not deleterious to the subject to be treated. The carrier may serve as a diluent, vehicle, excipient, or medium for the active ingredient. Some examples of suitable carriers include physiologically compatible buffers, such as Hank's solution, Ringer's solution, physiological saline buffer, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The pharmaceutical composition can additionally include lubricating agents such as talc, magnesium stearate, and mineral oil; wetting agents; emulsifying and suspending agents; preserving agents such as methyl- and propylhydroxy-benzoates; sweetening agents; and flavoring agents.

The pharmaceutical composition according to the invention can be in the form of tablets, pills, powders, lozenges, sachets, cachets, elixirs, suspensions, emulsions, solutions, syrups, soft and hard gelatin capsules, suppositories, sterile injectable solutions, and packaged powders.

The pharmaceutical composition of the invention may be delivered through any physiologically acceptable route such as orally, parentally (e.g. intramuscularly, intravenously, subcutaneously, interperitoneally), transdermally, rectally, by inhalation and the like. In one embodiment, the composition of the invention is orally administrated.

In some embodiments, the AXL inhibitor of the invention can be administered in combination with one or more known antiviral drug. Examples of the known antiviral drug include, but are not limited to, amantadine, rimantadine, oseltamivir, zanamivir, laninamivir, and peramivir. The AXL inhibitor of the invention and such antivial drugs can be administered either simultaneously or sequentially.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### 1. Materials and Methods

1.1 Cell Lines and Virus

The human lung epithelial carcinoma cell line A549 was maintained in F-12 medium (Gibco) supplemented with 10% fetal bovine serum (FBS, Gibco). The human embryonic kidney 293T and Madin-Darby canine kidney cells (MDCK) were cultured in Dulbecco's modified essential medium (DMEM, Gibco) supplemented with 10% FBS. The tetracycline-regulated expression 293 cell (T-REx-293, Invitrogen) was grown in DMEM containing 10% FBS and 5 μg/ml blasticidin (InvivoGen). Influenza A virus (IAV) (A/WSN/33, H1N1) strain was propagated in MDCK cell line in MEM alpha (Gibco) medium containing 0.5 μg/ml of L-(tosyl-amido-2-phenylethyl) chloromethyl ketone (TPCK)-trypsin (Pierce). Virus titers of IAV were determined by plaque-forming assays on MDCK cells.

1.2 Plaque Assay

Monolayer of MDCK cells (6×10⁵) were seeded in 6-well plates, and inoculated with a ten-fold dilution series of viral supernatant in MEM alpha medium (Gibco) containing 0.5 μg/ml TPCK-trypsin and incubated at 37° C. for 1 h. Then, the supernatant was removed and cells were overlaid with 3 ml of agarose medium DMEM containing 0.5% SeaKem LE agarose (Lonza) to each inoculated well. After 2 days, the cells were fixed with 3.7% formaldehyde and stained with 1% Crystal violet solution.

1.3 Plasmid Constructs

A pcDNA5/TO vector (Invitrogen) was used for the inducible expression of AXL. Human AXL (NM_{—}021913.4) were PCR amplified using the primers human AXL forward (5′-CCCAAGCTTGGGATGGCGTGGCGGTGCCCCAG) (SEQ ID NO: 3) and human AXL reverse (5′-ATAGTTTAGCGGCCGCTCAGGCACCATCCTCCTGCCCTGG) (SEQ ID NO: 4) and cloned into the HindIII and Not I sites of the pcDNA5/TO vector. Underlined nucleotides represent the HindIII and Not I sequence. For the expression of IAV proteins, cDNA fragments of IAV (WSN) encoding the individual viral proteins were subcloned to a hemagglutinin (HA) epitope-tagged pCAG vector backbone.

1.4 Establishment of Stable Cell Lines

The lentivirus vector pLKO.1, carrying the short hairpin RNA (shRNA) targeting human AXL (#1, TRCN0000000575 and #2, TRCN0000001040, 5′-CGAAATCCTCTATGTCAACAT (SEQ ID NO: 5), targeting the same nt 2451 to 2471 of the human AXL mRNA and negative control targeting LacZ (TRCN0000072224, 5′-CGCGATCGTAATCACCCGAGT (SEQ ID NO: 6), targeting the 3′UTR of the LacZ mRNA) were obtained from the Taiwan National RNAi Core Facility. To knock down human AXL expression, the A549 cells were transduced with shAXL lentivirus for 24 h and selected with puromycin (3 μg/ml, Sigma). For generation of inducible human AXL stable cell line, T-REx-293 cells were transfected with pcDNA5/TO encoding human AXL and selected with hygromycin (250 μg/ml, Roche) and blasticidin (5 μg/ml, InvivoGen) for 10 days. Individual colonies were picked and expanded in DMED containing 10% FBS, hygromycin and blasticidin.

1.5 Western Immunoblotting.

For Western immunoblotting, cells were lysed with M-PER mammalian extraction reagent (Thermo) containing a cocktail of protease inhibitors (Roche). The protein concentrations were determined by using the dye regent protein assay (Bio-Rad). Equal amounts of proteins were loaded and separated by SDS-PAGE and then transferred to a PVDF membrane (Hybond-C Super; Amersham/GE Healthcare). The PVDF membrane was blocked with skim milk in phosphate-buffered saline (PBS) with 0.1% Tween 20 (PBST) and subsequently incubated with primary antibody against various proteins, including mouse anti-influenza A nucleoprotein (NP) (1:3,000, Abcam), mouse anti-influenza A non-structural protein 1 (NS1) (1:1,000, Santa Cruz), goat anti-influenza A matrix protein 1 (M1) (1:1000, Santa Cruz), rabbit anti-AXL (1:1000, Cell signaling), rabbit anti-phospho-AXL (Tyr702) (Tyr779) (1:1,000, Cell signaling), mouse anti-actin (1:5,000, Millipore), rabbit anti-HA tag (1:5000, Santa Cruz). The blots then were reacted with a horseradish peroxidase-conjugated secondary antibody (1:3,000; Jackson ImmunoResearch) and developed using an enhanced chemiluminescence system (ECL; Millipore).

1.6 Transmission Electron Microscopy

T-RE-x-293 vector, AXL clone #1, and AXL #2 cells were seeded on ACLAR embedding film and treated with doxycycline (Dox, 1 μg/ml). Cells were infected with IAV at MOI of 0.01 for 24 h and then rinsed with 0.1 M sodium cacodylate buffer at 4° C. For the fixation, cells were kept at 4° C. throughout all the procedure. First, cells were fixed by 2.5% glutaraldehyde in 0.1 M sodium cacodylate buffer for 1 h and washed by 0.1 M sodium cacodylate buffer. Second, cells were postfixed by 1% osmium tetraoxide in 0.1 M sodium cacodylate buffer for 1 h and washed by water, and then stained with 1% uranyl acetate for 1 h. After staining, cells were washed by water and followed by dehydration in a graded ethanol series and embedding in resin. Then, samples were allowed to polymerize for 14 h at 70° C. and were cut into thin sections using ultramicroteme and collected on copper grids. The grids were observed with Tecnai G2 Spirit TWIN (FEI Company).

1.7 Quantitative RT-PCR

Total RNAs were extracted by using RNA isolation Kit (Roche) according to the manufacturer's protocol, and subjected to reverse transcription. For the detection of viral RNA, RT-PCR was performed by using the primer uni-12 (5′-AGCAAAAGCAGG) (SEQ ID NO: 7). For AXL and GAPDH detection, RT-PCR was performed by using the primer oligo-dT. The cDNA was reverse transcribed from 1.2 μg of RNA with primers using a ThermoScript RT kit (Invitrogen). The quantitative RT-PCR was performed with the Universal ProbeLibrary (UPL, Roche) with the indicated primers: forward IAV-NP: 5′-GATGGAGACTGATGGAGAACG (SEQ ID NO: 8) and reversed IAV-NP: 5′-TCATTTTTCCGACAGATGCTC (SEQ ID NO: 9) or forward AXL: 5′-CGTAACCTCCACCTGGTCTC (SEQ ID NO: 10) and reversed AXL: 5′-TCCCATCGTCTGACAGCA (SEQ ID NO: 11) or forward GAPDH: 5′-AGCCACATCGCTCAGACAC (SEQ ID NO: 12) and reversed GAPDH: 5′-GCCCAATACGACCAAATCC (SEQ ID NO: 13). For relative quantification, IAV-NP RNA and AXL RNA was measured with respect to GAPDH in each sample and the concentration was calculated from a standard curve.

1.8 Infection Inhibition Assay

For antibody blocking, A549 cells grown on 12-well plates were treated with a goat anti-AXL polyclonal antibody (25 μg/ml, R&D, against amino acid residues 33-440 of AXL, SEQ ID NO: 14) at the indicated times before, during, or post-infection with IAV (MOI of 0.2) for 17 h. As a control, medium alone (mock) and preimmune goat immunoglobulins (control Ab, SouthernBiotech), or sialidase (0.01 units/ml, Sigma), were used in a similar assay. The cell lysates were harvested for immunoblotting by antibodies against IAV-NP, -NS1, or actin.

For AXL kinase inhibitor blocking, A549 cells seeded on 12-well plates were treated with medium alone (mock), solvent control (DMSO), or 8 μM of R428 (1-(6,7-dihydro-5H-benzo[6,7]cyclohepta[1,2-c]pyridazin-3-yl)-N3-(7-(pyrrolidin-1-yl)-6,7,8,9-tetrahydro-5H-benzo[7]annulen-2-yl)-1H-1,2,4-triazole-3,5-diamine, Symansis) at the indicated times before, during, or post-infection with IAV (MOI of 0.5) for 8 h. The cell lysates were collected for Western blotting with antibodies against IAV-NP, -NS1, or actin.

1.9 Binding Assay

For antibody blocking virus attachment, the cells were pretreated with anti-AXL antibody and then adsorbed with the IAV at a MOI of 0.2 in the presence of antibody for 1 h at 4° C. After adsorption, the cells were washed for removing unbound viral particles and antibodies and further incubated for 17 h at 37° C. The cell lysates were harvested for Western blotting by antibodies against IAV-NP, -NS1, or actin.

We also used a published protocol for viral-binding assays (Eierhoff et al., 2009). The cells were harvested with cell scrapers and the amounts of adsorbed viruses on cell surface were determined by immunoblotting with antibody against virion-associated matrix M1 protein. The A549-shAXL clone #1, #2, and control shLacZ cells were adsorbed with IAV (MOI of 5) at 4° C. for 1 h, and a PBS wash (PBS, pH7.4) was performed to remove unattached virus before cell lysis. The attached virus particles were visualized with a M1 antibody by immunoblotting analysis.

1.10 Immunoprecipitation-Western Assay

T-RE-x-293 AXL cells were transfected with individual plasmids encoding HA-tagged NP, HA, NA, M2, or mCherry and also treated with Dox for induction of AXL expression. The whole-cell lysates were immunoprecipitated with anti-HA-agarose (Pierce) or anti-AXL (Cell signaling) overnight at 4° C. For immunoprecipitation by anti-AXL, the immunocomplex were further captured by Protein A/G (Roche) at 4° C. for 3 h. The precipitates were washed by wash buffer (1×TBS with 0.1% Tween-20) and proteins were analyzed by immunoblotting with antibodies against HA-tagged viral proteins and AXL.

### 2. Results

2.1 Human AXL Mediated IAV Infection

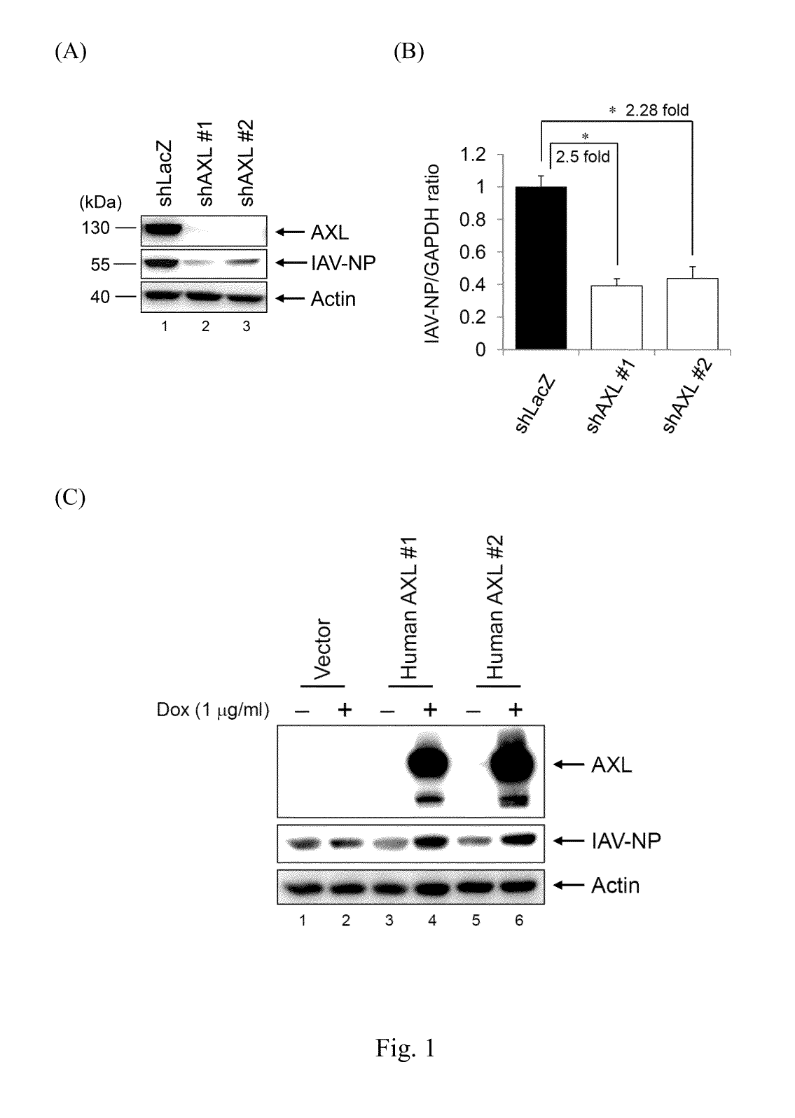

A549 cells were first deprived of their AXL expression by transduction with a lentivirus expressing a shRNA targeting the AXL, resulting in the A549-shAXL cell line. AXL expression was greatly decreased by shAXL clones #1 and #2 at protein levels as compared to those of shLacZ control cells (FIG. 1A). To address the role of AXL in IAV replication, A549-shAXL cells were tested for their ability to support virus replication. A549-shLacZ and shAXL cells were infected with influenza A virus (IAV) and viral nucleoprotein (NP) protein was measured by immunoblotting and viral RNA by quantitative RT-PCR (qRT-PCR). The replication of IAV was significantly reduced in A549-shAXL clones #1 and #2 cells (FIG. 1A). The RNA level of IAV was decreased by 2.5-and 2.28-fold in A549-shAXL clones #1 and #2 cells as compared to those of shLacZ control cells, respectively (FIG. 1B).

Next, we assessed whether the replication of IAV was enhanced in AXL over-expressing cells. We used an inducible promoter to specifically over-express AXL in 293T cells, which have been reported to lack AXL expression (Morizono et al., 2011). We cloned the human cDNA encoding the AXL protein and established inducible cell lines expressing the AXL protein in HEK T-RE-x-293 cells. Individual colonies of T-REx-293/human AXL clones #1 and #2 cells were picked and expanded. With Doxycycline (Dox) treatment, the human AXL protein was induced and detected by AXL antibody (FIG. 1C). To assess the possible enhancement of IAV infection by over-expression of AXL, the T-REx-293/human AXL clone #1 and #2 were cultured in the presence or absence of Dox and then infected with IAV. The replication of IAV was slightly enhanced in cells with AXL protein expression (FIG. 1C). The combination of the results of IAV replication in knockdown and over-expression of AXL suggests that AXL is involved in IAV replication.

2.2 AXL Blockage Greatly Reduced the IAV Infection During Virus Entry Stage

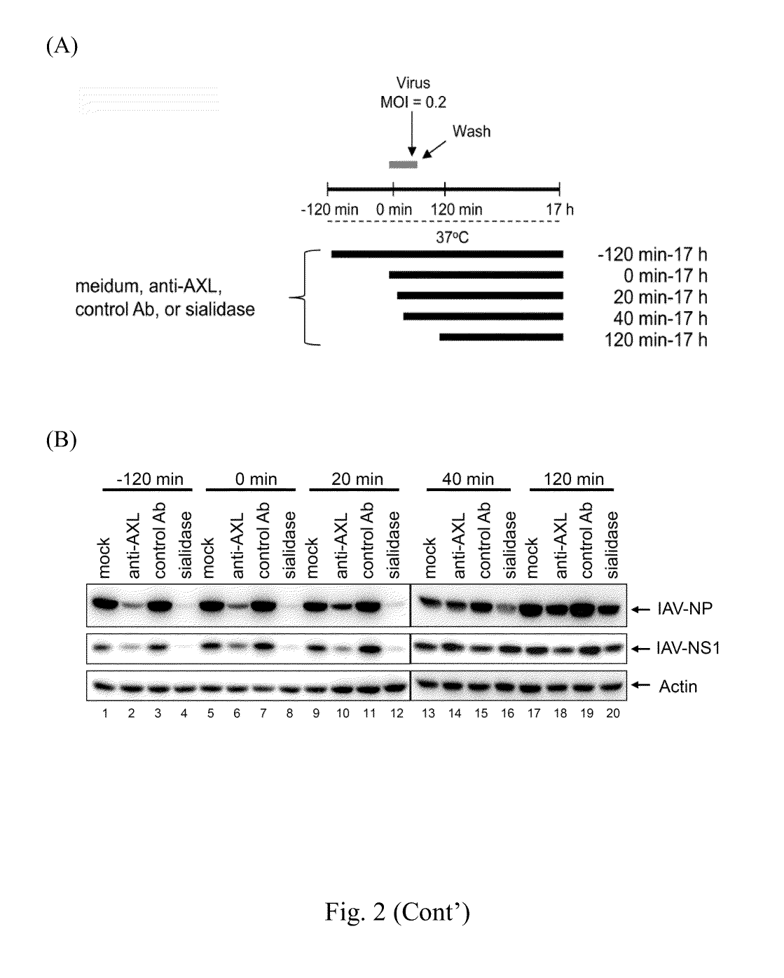

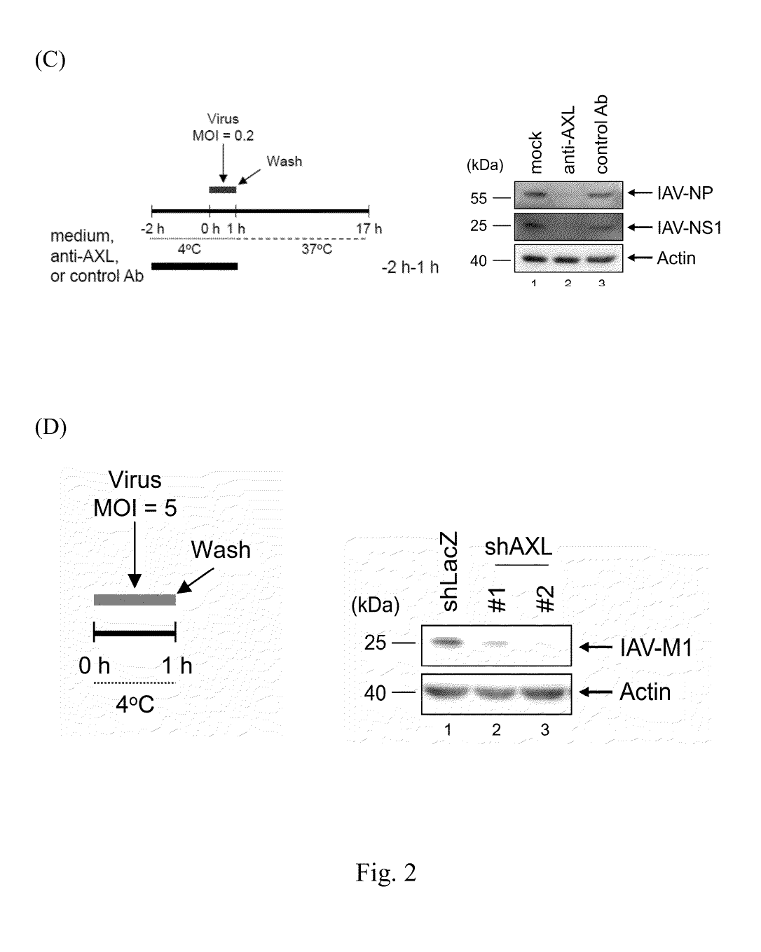

In this experiment, we proceeded to address whether blocking AXL with antibodies could affect the IAV entry process. The medium, AXL blocking antibody, preimmune control, or sialidase were added to virus-infected cells at different time points pre-, during, and post-viral infection and maintained throughout the viral life cycle (FIG. 2A). The treatment of A549 cells with exogenous sialidase efficiently removed surface sialic acid and abolished IAV infection (FIG. 2B, lanes 4, 8, and 12) as evident by reductions in viral NP and NS1 protein expression at 17 hours post-infection. Sialidase treatment was effective in inhibiting virus infection only when it was added before 20 minutes post-virus infection. Similarly, the anti-AXL antibody blocked IAV infection and replication only if it was added 40 min or earlier after the addition of virus (FIG. 2B, lanes 2, 6, 10, and 14). These results showed that AXL was involved in the very early stages of IAV infection, very similar to the functions of sialic acid. The blockage of virus infection during the early stage of infection could be due to failure of virus binding to the cell surface or failure of virus entry. To distinguish these two possibilities, we performed direct virus binding assay. Cells were incubated with virus in the presence of, anti-AXL antibody, or preimmune serum on ice for 1 hour. Earlier studies showed that virus could be attached but not internalized into cells at 4° C. (Eierhoff et al., 2009). The viral inoculates and antibodies were washed away, and the cells were further incubated until 17 h post-infection. The cell lysates were harvested for viral protein detection. The infectivity of IAV was blocked by anti-AXL antibody but not preimmune serum (FIG. 2C, lane 2). This experiment showed that the step of IAV infection affected by anti-AXL was most likely to be viral binding. To further demonstrate the involvement of AXL in IAV binding, we incubated the A549-shAXL and -shLacZ cells with virus at 4° C. for 1 h, washed the cells with PBS and then lysed cells to measure the amounts of virion-associated matrix protein (M1) remaining on the cell surface. As evidenced by reduced M1 signal, the mounts of IAV binding were significantly reduced by down-regulation of AXL expression (FIG. 2D, lanes 2 and 3). Our results suggest that AXL is a cell surface protein capable of binding IAV and is involved in IAV infection.

2.3 AXL Kinase Activity was Induced by Virus Binding and Required for Efficient IAV Uptake

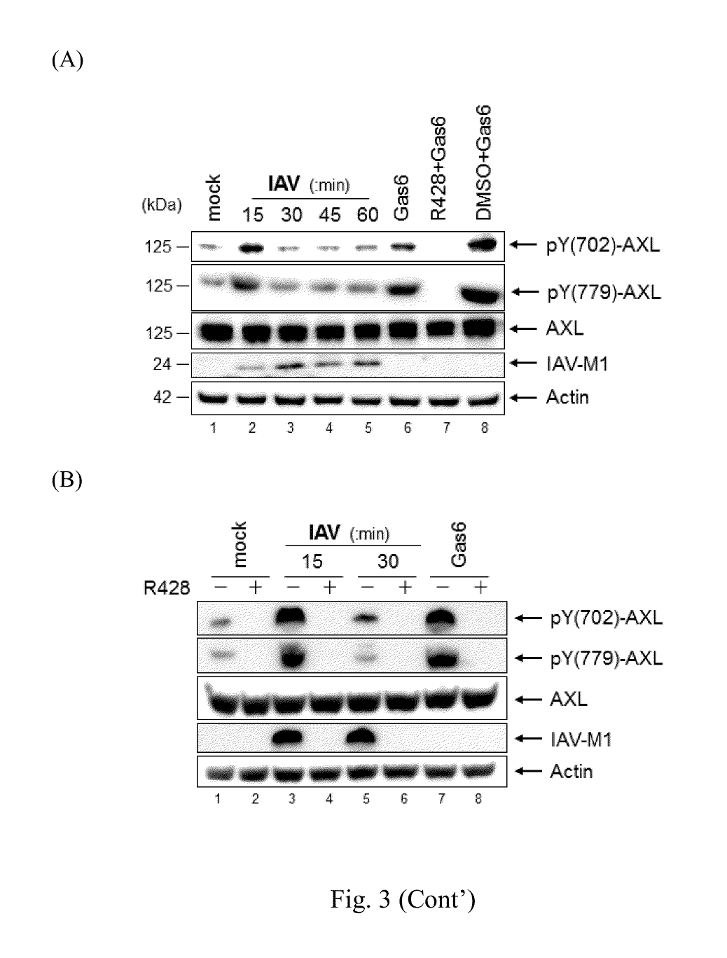

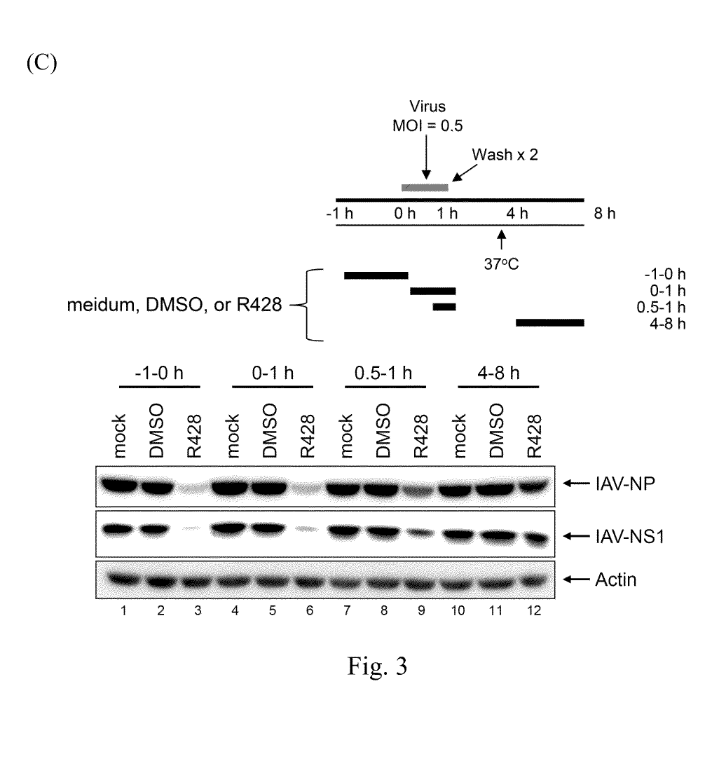

Since AXL is a receptor tyrosine kinase, its phosphor-tyrosine sites may provide some docking sites for some adaptors, which can further recruit downstream signaling molecules, or molecules regulating the endocytosis pathway. To demonstrate the biological significance of virus attachment to AXL, we determined whether virus binding could induce AXL kinase activation. The control experiment showed that phosphorylation of tyrosine 702 of AXL was induced by its ligand growth-arrest-specific gene 6 (Gas6) as reported (Linger et al., 2008) (FIG. 3A, lane 6). We found that tyrosine 702 and 779 became phosphorylated transiently in response to IAV attachment within 15 min post-infection, most of tyrosine phosphorylation was removed within 30 min (FIG. 3A, lanes 2 and 3). The virus was internalized within 30 min as detected by M1 in immunoblotting (FIG. 3A). The R428, a known AXL kinase inhibitor, has been reported to block autophosphorylation which stimulated by Gas6 (Holland et al., 2010) but not by DMSO control (FIG. 3A, lanes 7 and 8, see also FIG. 3B). To verify whether the kinase activity of AXL regulates the IAV uptake into host cells, A549 cells were treated with R428 during different stages of viral infection in a time-of-addition assay (FIG. 3C). R428 inhibited IAV replication as evident by reduction of NP and NS1 protein expression when added before or during the first hour, but not later (FIG. 3C, lanes 3, 6, and 9). We concluded that the AXL kinase was activated by IAV attachment and the kinase activity was involved in the initial IAV uptake into cells.

2.4 AXL Facilitate IAV Attachment in a Sialic Acid Dependent Manner

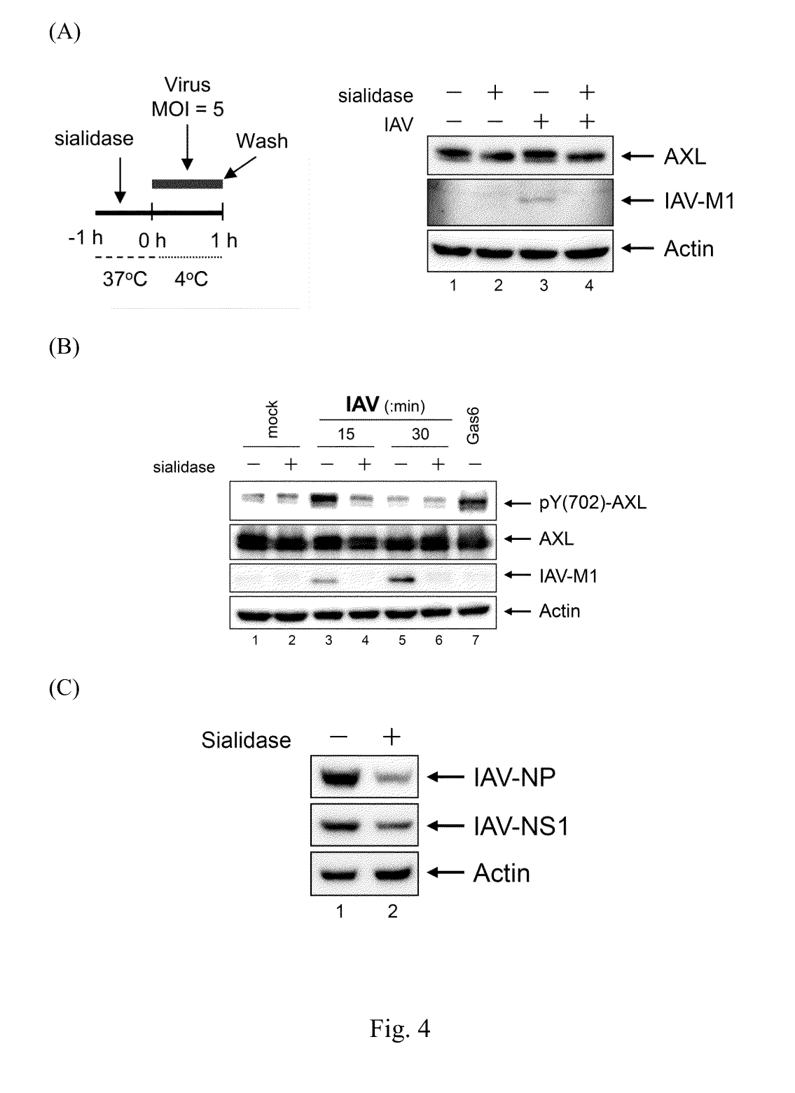

We next addressed whether AXL-promoted IAV infection is sialic acid dependent. In the cells over-expressing AXL, the binding of IAV particles to cell surface were completely blocked by sialidase pre-treatment (FIG. 4A, lane 3 and 4). The IAV-induced AXL phosphorylation at 15 min post-infection, was also blocked by sialidase pre-treatment (FIG. 4B, lanes 3 and 4). Correspondingly, the IAV replication was significantly reduced upon pre-treatment with sialidase (FIG. 4C). Together, these data suggest that AXL needs sialic acid to facilitate IAV attachment.

2.5 Identification of AXL Interacting with IAV Envelope Proteins

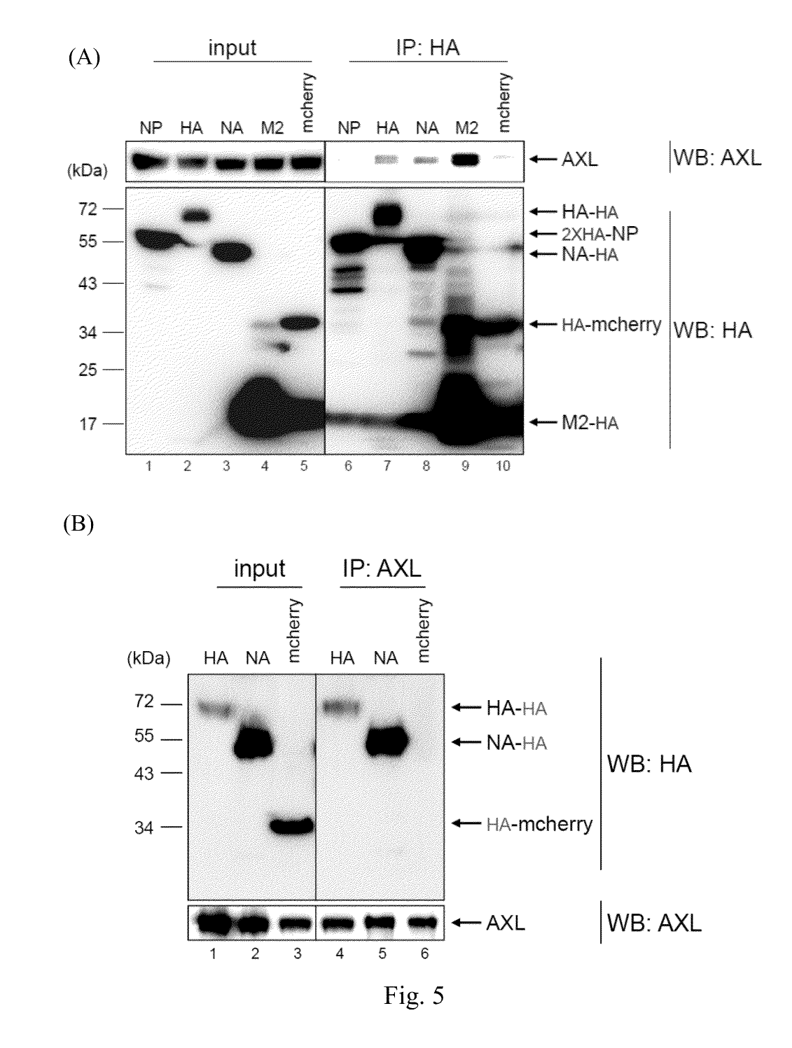

In our study, we demonstrated that AXL is a dual function receptor. AXL can facilitate IAV attachment and restrict virus release through tethering virions on plasma membrane. These results imply that there might be a direct interaction between IAV viral particles and AXL. To further assess whether the AXL interacts with envelope glycoproteins or matrix proteins of viral particles, T-RE-x-293/human AXL cells were transfected with individual plasmids encoding HA-tagged IAV-NP, HA, NA, or M2 viral proteins and also treated with Dox for induction of AXL expression. The viral protein-binding ability of AXL was analyzed by immunoprecipitation with antibody specific against HA-tag or AXL. The AXL was co-immune precipitated with envelope viral proteins, such as HA, NA, and M2, but not by NP and mcherry proteins (FIG. 5A, lanes 6-10). The HA, NA, and M2 were also precipitated by AXL (FIG. 5B, lanes 4, 5, 10, and 11). These results indicated a specific interaction between AXL and influenza A viral envelope proteins.

### 3. Summary

Virus binding to a cell surface receptor and its subsequent entry into host cells are critical steps determining viral species specificity and organ tropism. However, there are few therapeutics which target virus entry. Influenza virus A (IAV) is known to use sialic acid-containing molecules as the primary receptor. Its possible use of other receptors will expand the viral host range and offer potential new targets for antiviral therapy. By using an RNAi (interfering RNA) library screening to search for cellular factors required for influenza virus infection and replication, we identified a receptor tyrosine kinase anexelekto (AXL) as a cellular factor required for IAV infection. We found that IAV replication was suppressed by knockdown of AXL in A549 cells and enhanced by over-expression of AXL, indicating a positive role for AXL in IAV infection. Furthermore, treatment with a polyclonal antibody against AXL during virus binding stage (first 20 min), but not at the later stage, completely blocked virus infection even in the presence of sialic acid receptors. Under the same condition, the sialidase treatment completely blocked IAV infection despite the presence of AXL, indicating that AXL is a sialic acid-dependent receptor for IAV. Direct virus binding assay showed that IAV binding was decreased by knockdown of AXL, and that binding is dependent on both AXL and sialic acid. The IAV infection induced transient AXL phosphorylation, and inhibition of AXL kinase activity by a specific inhibitor leads to impaired IAV uptake into cells. Co-immunoprecipitation assay showed that AXL interacted with the viral ion channel protein M2 and the both envelope proteins neuraminidase (NA), and hemagglutinin (HA). In conclusion, AXL is found as a novel receptor for IAV infection, which offers a new target for antiviral therapy.

### Sequence Information

Human AXL NP_068713.2 894 a.a.
(SEQ ID NO: 2)
MAWRCPRMGRVPLAWCLALCGWACMAPRGTQAEESPFVGNPGNITGA
RGLTGTLRCQLQVQGEPPEVHWLRDGQILELADSTQTQVPLGEDEQD
DWIVVSQLRITSLQLSDTGQYQCLVFLGHQTFVSQPGYVGLEGLPYF
LEEPEDRTVAANTPFNLSCQAQGPPEPVDLLWLQDAVPLATAPGHGP
QRSLHVPGLNKTSSFSCEAHNAKGVTTSRTATITVLPQQPRNLHLVS
RQPTELEVAWTPGLSGIYPLTHCTLQAVLSDDGMGIQAGEPDPPEEP
LTSQASVPPHQLRLGSLHPHTPYHIRVACTSSQGPSSWTHWLPVETP
EGVPLGPPENISATRNGSQAFVHWQEPRAPLQGTLLGYRLAYQGQDT
PEVLMDIGLRQEVTLELQGDGSVSNLTVCVAAYTAAGDGPWSLPVPL
EAWRPGQAQPVHQLVKEPSTPAFSWPWWYVLLGAVVAAACVLILALF
LVHRRKKETRYGEVFEPTVERGELVVRYRVRKSYSRRTTEATLNSLG
ISEELKEKLRDVMVDRHKVALGKTLGEGEFGAVMEGQLNQDDSILKV
AVKTMKIAICTRSELEDFLSEAVCMKEFDHPNVMRLIGVCFQGSERE
SFPAPVVILPFMKHGDLHSFLLYSRLGDQPVYLPTQMLVKFMADIAS
GMEYLSTKRFIHRDLAARNCMLNENMSVCVADFGLSKKIYNGDYYRQ
GRIAKMPVKWIAIESLADRVYTSKSDVWSFGVTMWEIATRGQTPYPG
VENSEIYDYLRQGNRLKQPADCLDGLYALMSRCWELNPQDRPSFTEL
REDLENTLKALPPAQEPDEILYVNMDEGGGYPEPPGAAGGADPPTQP
DPKDSCSCLTAAEVHPAGRYVLCPSTTPSPAQPADRGSPAAPGQEDGA
Mouse AXL NP_033491 888 a.a.
(SEQ ID NO: 15)
MGRVPLAWWLALCCWGCAAHKDTQTEAGSPFVGNPGNITGARGLTGT
LRCELQVQGEPPEVVWLRDGQILELADNTQTQVPLGEDWQDEWKVVS
QLRISALQLSDAGEYQCMVHLEGRTFVSQPGFVGLEGLPYFLEEPED
KAVPANTPFNLSCQAQGPPEPVTLLWLQDAVPLAPVTGHSSQHSLQT
PGLNKTSSFSCEAHNAKGVTTSRTATITVLPQRPHHLHVVSRQPTEL
EVAWTPGLSGIYPLTHCNLQAVLSDDGVGIWLGKSDPPEDPLTLQVS
VPPHQLRLEKLLPHTPYHIRISCSSSQGPSPWTHWLPVETTEGVPLG
PPENVSAMRNGSQVLVRWQEPRVPLQGTLLGYRLAYRGQDTPEVLMD
IGLTREVTLELRGDRPVANLTVSVTAYTSAGDGPWSLPVPLEPWRPG
QGQPLHHLVSEPPPRAFSWPWWYVLLGALVAAACVLILALFLVHRRK
KETRYGEVFEPTVERGELVVRYRVRKSYSRRTTEATLNSLGISEELK
EKLRDVMVDRHKVALGKTLGEGEFGAVMEGQLNQDDSILKVAVKTMK
IAICTRSELEDFLSEAVCMKEFDHPNVMRLIGVCFQGSDREGFPEPV
VILPFMKHGDLHSFLLYSRLGDQPVFLPTQMLVKFMADIASGMEYLS
TKRFIFIRDLAARNCMLNENMSVCVADFGLSKKIYNGDYYRQGRIAK
MPVKWIAIESLADRVYTSKSDVWSFGVTMWEIATRGQTPYPGVENSE
IYDYLRQGNRLKQPVDCLDGLYALMSRCWELNPRDRPSFAELREDLE
NTLKALPPAQEPDEILYVNMDEGGSHLEPRGAAGGADPPTQPDPKDS
CSCLTAADVHSAGRYVLCPSTAPGPTLSADRGCPAPPGQEDGA

### REFERENCE

Air, G. M., and W. G. Laver. 1989. The neuraminidase of influenza virus. Proteins 6:341-56.
Angelillo-Scherrer, A., P. de Frutos, C. Aparicio, E. Melis, P. Savi, F. Lupu, J. Arnout, M. Dewerchin, M. Hoylaerts, J. Herbert, D. Cohen, B. Dahlback, and P. Carmeliet. 2001. Deficiency or inhibition of Gas6 causes platelet dysfunction and protects mice against thrombosis. Nat Med 7:215-21.
Braunger, J., L. Schleithoff, A. S. Schulz, H. Kessler, R. Lammers, A. Ullrich, C. R. Bartram, and J. W. Janssen. 1997. Intracellular signaling of the Ufo/Axl receptor tyrosine kinase is mediated mainly by a multi-substrate docking-site. Oncogene 14:2619-31.
Brindley, M. A., C. L. Hunt, A. S. Kondratowicz, J. Bowman, P. L. Sinn, P. B. McCray, Jr., K. Quinn, M. L. Weller, J. A. Chiorini, and W. Maury. 2011. Tyrosine kinase receptor Axl enhances entry of Zaire ebolavirus without direct interactions with the viral glycoprotein. Virology 415:83-94.
Eierhoff, T., S. Ludwig, and C. Ehrhardt. 2009. The influenza A virus matrix protein as a marker to monitor initial virus internalisation. Biol Chem 390:509-15.
Fridell, Y. W., Y. Jin, L. A. Quilliam, A. Burchert, P. McCloskey, G. Spizz, B. Varnum, C. Der, and E. T. Liu. 1996. Differential activation of the Ras/extracellular-signal-regulated protein kinase pathway is responsible for the biological consequences induced by the Axl receptor tyrosine kinase. Mol Cell Biol 16:135-45.
Holland, S. J., A. Pan, C. Franci, Y. Hu, B. Chang, W. Li, M. Duan, A. Torneros, J. Yu, T. J. Heckrodt, J. Zhang, P. Ding, A. Apatira, J. Chua, R. Brandt, P. Pine, D. Goff, R. Singh, D. G. Payan, and Y. Hitoshi. 2010. R428, a selective small molecule inhibitor of Axl kinase, blocks tumor spread and prolongs survival in models of metastatic breast cancer. Cancer Res 70:1544-54.
Kogure, T., T. Suzuki, T. Takahashi, D. Miyamoto, K. I. Hidari, C. T. Guo, T. Ito, Y. Kawaoka, and Y. Suzuki. 2006. Human trachea primary epithelial cells express both sialyl(alpha2-3)Gal receptor for human parainfluenza virus type 1 and avian influenza viruses, and sialyl(alpha2-6)Gal receptor for human influenza viruses. Glycoconj J 23:101-6.
Lakadamyali, M., M. J. Rust, H. P. Babcock, and X. Zhuang. 2003. Visualizing infection of individual influenza viruses. Proc Natl Acad Sci USA 100:9280-5.
Lakadamyali, M., M. J. Rust, and X. Zhuang. 2004. Endocytosis of influenza viruses. Microbes Infect 6:929-36.
Linger, R. M., A. K. Keating, H. S. Earp, and D. K. Graham. 2008. TAM receptor tyrosine kinases: biologic functions, signaling, and potential therapeutic targeting in human cancer. Adv Cancer Res 100:35-83.
Lu, Q., and G. Lemke. 2001. Homeostatic regulation of the immune system by receptor tyrosine kinases of the Tyro 3 family. Science 293:306-11.
Luo, M. 2012. Influenza virus entry. Adv Exp Med Biol 726:201-21.
Matrosovich, M. N., T. Y. Matrosovich, T. Gray, N. A. Roberts, and H. D. Klenk. 2004. Neuraminidase is important for the initiation of influenza virus infection in human airway epithelium. J Virol 78:12665-7.
Mercer, J. 2011. Viral apoptotic mimicry party: P.S. Bring your own Gas6. Cell Host Microbe 9:255-7.
Morizono, K., Y. Xie, T. Olafsen, B. Lee, A. Dasgupta, A. M. Wu, and I. S. Chen. 2011. The soluble serum protein Gas6 bridges virion envelope phosphatidylserine to the TAM receptor tyrosine kinase Axl to mediate viral entry. Cell Host Microbe 9:286-98.
Neubauer, A., A. Fiebeler, D. K. Graham, J. P. O'Bryan, C. A. Schmidt, P. Barckow, S. Serke, W. Siegert, H. R. Snodgrass, D. Huhn, and et al. 1994. Expression of axl, a transforming receptor tyrosine kinase, in normal and malignant hematopoiesis. Blood 84:1931-41.
Nielsen-Preiss, S. M., M. P. Allen, M. Xu, D. A. Linseman, J. E. Pawlowski, R. J. Bouchard, B. C. Varnum, K. A. Heidenreich, and M. E. Wierman. 2007. Adhesion-related kinase induction of migration requires phosphatidylinositol-3-kinase and ras stimulation of rac activity in immortalized gonadotropin-releasing hormone neuronal cells. Endocrinology 148:2806-14.
O'Bryan, J. P., R. A. Frye, P. C. Cogswell, A. Neubauer, B. Kitch, C. Prokop, R. Espinosa, 3rd, M. M. Le Beau, H. S. Earp, and E. T. Liu. 1991. axl, a transforming gene isolated from primary human myeloid leukemia cells, encodes a novel receptor tyrosine kinase. Mol Cell Biol 11:5016-31.
Ohuchi, M., N. Asaoka, T. Sakai, and R. Ohuchi. 2006. Roles of neuraminidase in the initial stage of influenza virus infection. Microbes Infect 8:1287-93.
Palese, P., K. Tobita, M. Ueda, and R. W. Compans. 1974. Characterization of temperature sensitive influenza virus mutants defective in neuraminidase. Virology 61:397-410.
Rust, M. J., M. Lakadamyali, F. Zhang, and X. Zhuang. 2004. Assembly of endocytic machinery around individual influenza viruses during viral entry. Nat Struct Mol Biol 11:567-73.
Shimojima, M., A. Takada, H. Ebihara, G. Neumann, K. Fujioka, T. Irimura, S. Jones, H. Feldmann, and Y. Kawaoka. 2006. Tyro3 family-mediated cell entry of Ebola and Marburg viruses. J Virol 80:10109-16.
Skehel, J. J., and D. C. Wiley. 2000. Receptor binding and membrane fusion in virus entry: the influenza hemagglutinin. Annu Rev Biochem 69:531-69.
Stitt, T. N., G. Conn, M. Gore, C. Lai, J. Bruno, C. Radziejewski, K. Mattsson, J. Fisher, D. R. Gies, P. F. Jones, and et al. 1995. The anticoagulation factor protein S and its relative, Gas6, are ligands for the Tyro 3/Axl family of receptor tyrosine kinases. Cell 80:661-70.
Su, B., S. Wurtzer, M. A. Rameix-Welti, D. Dwyer, S. van der Werf, N. Naffakh, F. Clavel, and B. Labrosse. 2009. Enhancement of the influenza A hemagglutinin (HA)-mediated cell-cell fusion and virus entry by the viral neuraminidase (NA). PLoS One 4:e8495.
Suzuki, T., A. Portner, R. A. Scroggs, M. Uchikawa, N. Koyama, K. Matsuo, Y. Suzuki, and T. Takimoto. 2001. Receptor specificities of human respiroviruses. J Virol 75:4604-13.
Varnum, B. C., C. Young, G. Elliott, A. Garcia, T. D. Bartley, Y. W. Fridell, R. W. Hunt, G. Trail, C. Clogston, R. J. Toso, and et al. 1995. Axl receptor tyrosine kinase stimulated by the vitamin K-dependent protein encoded by growth-arrest-specific gene 6. Nature 373:623-6.
Wang, H., S. Chen, Y. Chen, H. Wu, H. Tang, W. Xiong, J. Ma, Y. Ge, Q. Lu, and D. Han. 2007. The role of Tyro 3 subfamily receptors in the regulation of hemostasis and megakaryocytopiesis. Haematologica 92:643-50.
Weinger, J. G., P. Gohari, Y. Yan, J. M. Backer, B. Varnum, and B. Shafit-Zagardo. 2008. In brain, Axl recruits Grb2 and the p85 regulatory subunit of PI3 kinase; in vitro mutagenesis defines the requisite binding sites for downstream Akt activation. J Neurochem 106:134-46.
Wimmel, A., D. Glitz, A. Kraus, J. Roeder, and M. Schuermann. 2001. Axl receptor tyrosine kinase expression in human lung cancer cell lines correlates with cellular adhesion. Eur J Cancer 37:2264-74.
Zheng, Y., L. Zhang, Q. Lu, X. Wang, and F. Yu. 2009. NGF-induced Tyro3 and Axl function as survival factors for differentiating PC12 cells. Biochem Biophys Res Commun 378:371-5.

What is claimed is:

1. A method for treatment or prevention of an influenza virus type A infection which comprises administering to a subject a therapeutically effective amount of an anexelekto (AXL) inhibitor to a subject.

2. The method of claim 1, wherein said influenza virus type A is H1N1 virus.

3. The method of claim 1, wherein the AXL inhibitor is selected from the group consisting of a small molecule organic compound, an antibody and a polynucleotide.

4. The method of claim 1, wherein the AXL inhibitor is 1-(6,7-dihydro-5H-benzo[6,7]cyclohepta[1,2-c]pyridazin-3-yl)-N3-(7-(pyrrolidin-1-yl)-6,7,8,9-tetrahydro-5H-benzo[7]annulen-2-yl)-1H-1,2,4-triazole-3,5-diamine.

5. The method of claim 1, wherein the AXL inhibitor is an antibody that specifically binds to AXL.

6. The method of claim 1, wherein the AXL inhibitor is a polynucleotide selected from the group consisting of a short interfering RNA (siRNA), synthetic hairpin RNA (shRNA) or anti-sense nucleic acids.

7. The method of claim 1 wherein said subject is a mammal.

8. The method of claim 1 wherein said subject is a human.

9. The method of claim 1 wherein the AXL inhibitor is administered prior to or after the virus infection.

10. The method of claim 1 wherein the AXL inhibitor is administered within 12 hours, one day, two days, three days, four days or five days after the virus infection.


SEQUENCE LISTING (15 sequences)

SEQ ID NO: 1 - DNA, 4750 bp, Homo sapiens
gtgagggaaggaggcaggggtgctgagaaggcggctgctgggcagagccggtggcaagggcctcccctgccgctgtgccaggcaggcagtgccaaatccggggagcctggagctggggggagggccggggacagcccggccctgccccctcccccgctgggagcccaacaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagggcaagcacagccagtccaccagctggtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactggggaaaactccaccttcccactttcccaccccacgccttatccccacttgcagccctgtcttcctacctatcccacctccatcccagacaggtccctccccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaggcctactttttttttttttttttttttttttttttttgcgatagagtctcactgtgtcacccaggctggagtgcagtggtgcaatctcgcctcactgcaaccttcacctaccgagttcaagtgattttcctgccttggcctcccaagtagctgggattacaggtgtgtgccaccacacccggctaatttttatatttttagtagagacagggtttcaccatgttggccaggctggtctaaaactcctgacctcaagtgatctgcccacctcagcctcccaaagtgctgagattacaggcatgagccactgcactcaaccttaagacctactgttctaaagctctgacattatgtggttttagattttctggttctaacatttttgataaagcctcaaggttttaggttctaaagttctaagattctgattttaggagctaaggctctatgagtctagatgtttattcttctagagttcagagtccttaaaatgtaagattatagattctaaagattctatagttctagacatggaggttctaaggcctaggattctaaaatgtgatgttctaaggctctgagagtctagattctctggctgtaaggctctagatcataaggcttcaaaatgttatcttctcaagttctaagattctaatgatgatcaattatagtttctgaggctttatgataatagattctcttgtataagatcctagatcctaagggtcgaaagctctagaatctgcaattcaaaagttccaagagtctaaagatggagtttctaaggtccggtgttctaagatgtgatattctaagacttactctaagatcttagattctctgtgtctaagattctagatcagatgctccaagattctagatgattaaataagattctaacggtctgttctgtttcaaggcactctagattccattggtccaagattccggatcctaagcatctaagttataagactctcacactcagttgtgactaactagacaccaaagttctaataatttctaatgttggacacctttaggttctttgctgcattctgcctctctaggaccatggttaagagtccaagaatccacatttctaaaatcttatagttctaggcactgtagttctaagactcaaatgttctaagtttctaagattctaaaggtccacaggtctagactattaggtgcaatttcaaggttctaaccctatactgtagtattctttggggtgcccctctccttcttagctatcattgcttcctcctccccaactgtgggggtgtgcccccttcaagcctgtgcaatgcattagggatgcctcctttcccgcaggggatggacgatctcccacctttcgggccatgttgcccccgtgagccaatccctcaccttctgagtacagagtgtggactctggtgcctccagaggggctcaggtcacataaaactttgtatatcaacgagaaaaaaaa

SEQ ID NO: 2 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 3 - DNA, 32 bp, Artificial Sequence
  human AXL forward primer
cccaagcttgggatggcgtggcggtgccccag

SEQ ID NO: 4 - DNA, 40 bp, Artificial Sequence
  human AXL reverse primer
atagtttagcggccgctcaggcaccatcctcctgccctgg

SEQ ID NO: 5 - DNA, 21 bp, Artificial Sequence
  oligonucletotide for shRNA targeting human AXL
cgaaatcctctatgtcaacat

SEQ ID NO: 6 - DNA, 21 bp, Artificial Sequence
  oligonucleotide of shRNA targeting LacZ
cgcgatcgtaatcacccgagt

SEQ ID NO: 7 - DNA, 12 bp, Artificial Sequence
  primer uni-12
agcaaaagcagg

SEQ ID NO: 8 - DNA, 21 bp, Artificial Sequence
  forward IAV-NP
gatggagactgatggagaacg

SEQ ID NO: 9 - DNA, 21 bp, Artificial Sequence
  reverse IAV-NP
tcatttttccgacagatgctc

SEQ ID NO: 10 - DNA, 20 bp, Artificial Sequence
  forward AXL
cgtaacctccacctggtctc

SEQ ID NO: 11 - DNA, 18 bp, Artificial Sequence
  reverse AXL
tcccatcgtctgacagca

SEQ ID NO: 12 - DNA, 19 bp, Artificial Sequence
  forward GAPDH
agccacatcgctcagacac

SEQ ID NO: 13 - DNA, 19 bp, Artificial Sequence
  reversed GAPDH
gcccaatacgaccaaatcc

SEQ ID NO: 14 - protein, 408 aa, Homo sapiens
EESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKE

SEQ ID NO: 15 - protein, 888 aa, Mus musculus
MGRVPLAWWLALCCWGCAAHKDTQTEAGSPFVGNPGNITGARGLTGTLRCELQVQGEPPEVVWLRDGQILELADNTQTQVPLGEDWQDEWKVVSQLRISALQLSDAGEYQCMVHLEGRTFVSQPGFVGLEGLPYFLEEPEDKAVPANTPFNLSCQAQGPPEPVTLLWLQDAVPLAPVTGHSSQHSLQTPGLNKTSSFSCEAHNAKGVTTSRTATITVLPQRPHHLHVVSRQPTELEVAWTPGLSGIYPLTHCNLQAVLSDDGVGIWLGKSDPPEDPLTLQVSVPPHQLRLEKLLPHTPYHIRISCSSSQGPSPWTHWLPVETTEGVPLGPPENVSAMRNGSQVLVRWQEPRVPLQGTLLGYRLAYRGQDTPEVLMDIGLTREVTLELRGDRPVANLTVSVTAYTSAGDGPWSLPVPLEPWRPGQGQPLHHLVSEPPPRAFSWPWWYVLLGALVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSDREGFPEPVVILPFMKHGDLHSFLLYSRLGDQPVFLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPVDCLDGLYALMSRCWELNPRDRPSFAELREDLENTLKALPPAQEPDEILYVNMDEGGSHLEPRGAAGGADPPTQPDPKDSCSCLTAADVHSAGRYVLCPSTAPGPTLSADRGCPAPPGQEDGA